(19) United States
(12) Patent Application Publication
(10) Pub. No.: US 2004/0198981 A1
(43) Pub. Date: Oct. 7, 2004

(54) Tricyclic dihydroquinoline derivatives,method for preparing same and pharmaceutical compositions containing same

(76) Inventors: Henri-Philippe Husson, Chevreuse (FR); Sylviane Giorgi-Renault, Paris (FR); Stephanie Desbene, Paris (FR); John Hickman, Paris (FR); Alain Pierre, Les Alluets Le Roi (FR); Laurence Kraus-Berthier, Colombes (FR); Bruno Pfeiffer, Saint Leu La Foret (FR); Pierre Renard, Le Chesnay (FR)

Correspondence Address: THE FIRM OF HUESCHEN AND SAGE, 500 COLUMBIA PLAZA, 350 EAST MICHIGAN AVENUE, KALAMAZOO, MI, 49007, US

(21) Appl. No.: 10/477,258

(22) PCT Filed: May 22, 2002
(86) PCT No.: PCT/FR02/01715

(30) Foreign Application Priority Data
May 23, 2001 (FR) 01/06792

(51) Int. Cl. C07D 491/02; C07D 498/02; C07D 471/02

(52) U.S. Cl. 546/80; 546/81; 546/82

(57) ABSTRACT

Compound of formula (I):

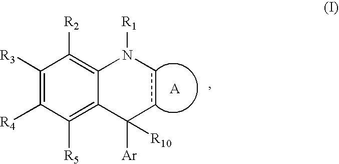

wherein:

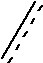

represents a single or double bond,

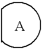

represents a ring system selected from

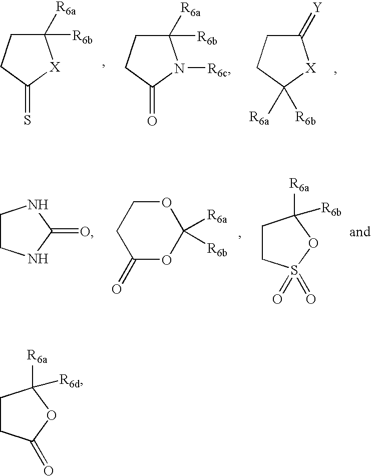

R₆ₐ, R_{6b}, R_{6c}, R_{6d}, X and Y are as defined in the description,

R₁ represents a hydrogen atom or a group selected from aryl, heteroaryl, cycloalkyl, optionally substituted alkyl, and COR₈ wherein R₈ is as defined in the description,

R₂, R₃, R₄ and R₅, which may be the same or different, each represent:

a hydrogen atom,

a halogen atom,

an alkyl group,.

an alkoxy group,

a hydroxy group,

a polyhaloalkyl group,

a nitro group,

an optionally substituted amino group,

a group of formula

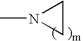

wherein m represents an integer such that 1≦m≦4,

or R₂ with R₃, or R₃ with R₄, or R₄ with R₅, form, together with the carbon atoms carrying them, an optionally substituted, 5- to 12-membered, mono- or bi-cyclic, aromatic or non-aromatic group optionally containing 1 or 2 hetero atoms selected from O, S and N,

R₁₀ represents a hydrogen atom or an alkyl group,

Ar represents an aryl, heteroaryl or arylalkyl group,

its optical isomers, addition salts thereof with a pharmaceutically acceptable acid, and hydrates and solvates thereof.

The present invention relates to new tricyclic dihydroquinoline compounds, to a process for their preparation, to pharmaceutical compositions containing them and to their use as anti-cancer agents.

Anti-cancer therapeutic requirements call for the constant development of new anti-tumour agents with the aim of obtaining medicaments that are simultaneously more active and better tolerated.

Besides the fact that the compounds of the invention are new, they possess very valuable anti-tumour properties.

Compounds having a closely related structure have been described in the literature, especially furo[3,4-b]quinolin-1-one compounds as anti-osteoporotic agents (patent specification EP 0 634 169).

More specifically, the present invention relates to compounds of formula (I):

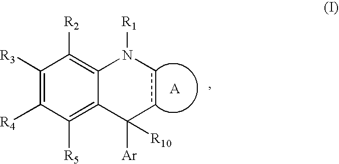

wherein:

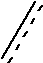

represents a single or double bond,

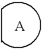

represents a ring system selected from

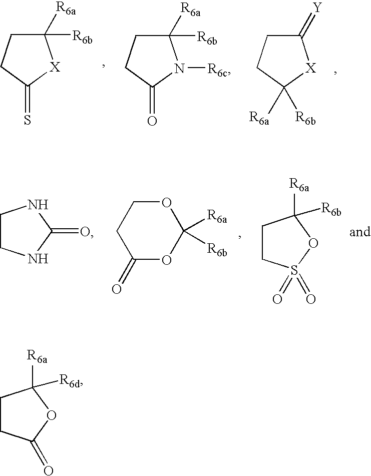

R₆ₐ, R_{6b} and R_{6c}, which may be the same or different, each represent a hydrogen atom or a linear or branched (C₁-C₆)alkyl group,

R_{6d}represents a linear or branched (C₁-C₆)alkyl group when R₁₀ represents a hydrogen atom, and a group selected from hydrogen and linear or branched (C₁-C₆)alkyl when R₁₀ represents a linear or branched (C₁-C₆)alkyl group,

X represents an oxygen or sulphur atom or a group NR_{6c}wherein R_{6c} represents a hydrogen atom or a linear or branched (C₁-C₆)alkyl group,

Y represents an oxygen or sulphur atom,

R₁ represents a hydrogen atom or a group selected from:

aryl,

heteroaryl,

(C₃-C₈)cycloalkyl,

linear or branched (C₁-C₆)alkyl optionally substituted by an aryl group, by a heteroaryl group, by a hydroxy group, by a linear or branched (C₁-C₆)alkoxy group, or by a group of formula NR₇ₐR_{7b} wherein R₇ₐ and R_{7b}, which may be the same or different, each represent a linear or branched (C₁-C₆)alkyl group optionally substituted by a hydroxy group or an amino group (itself optionally substituted by one or two linear or branched (C₁-C₆)alkyl groups), or R₇ₐ and R_{7b}, together with the nitrogen atom carrying them, form a nitrogen-containing heterocycle,

and COR₈ wherein R₈ represents a group:

aryl,

linear or branched (C₁-C₆)alkyl (optionally substituted by a group of formula NR₇ₐR_{7b} wherein R₇ₐ and R_{7b}, which may be the same or different, each represent a linear or branched (C₁-C₆)alkyl group optionally substituted by a hydroxy group or an amino group (itself optionally substituted by one or two linear or branched (C₁-C₆)alkyl groups), or R₇ₐ and R_{7b}, together with the nitrogen atom carrying them, form a nitrogen-containing heterocycle),

amino optionally substituted by one or more aryl groups, heteroaryl groups, or linear or branched (C₁-C₆)alkyl groups optionally substituted by a group of formula NR₇ₐR_{7b} wherein R₇ₐ and R_{7b}, which may be the same or different, each represent a linear or branched (C₁-C₆)alkyl group optionally substituted by a hydroxy group or an amino group (itself optionally substituted by one or two linear or branched (C₁-C₆)alkyl groups), or R₇ₐ and R_{7b}, together with the nitrogen atom carrying them, form a nitrogen-containing heterocycle,

or OR₉ wherein R₉ represents a hydrogen atom or an aryl group, or a linear or branched (C₁-C₆)alkyl group optionally substituted by a group of formula NR₇ₐR_{7b} wherein R₇ₐ and R_{7b}, which may be the same or different, each represent a linear or branched (C₁-C₆)alkyl group optionally substituted by a hydroxy group or an amino group (itself optionally substituted by one or two linear or branched (C₁-C₆)alkyl groups), or R₇ₐ and R_{7b}, together with the nitrogen atom carrying them, form a nitrogen-containing heterocycle,

R₂, R₃, R₄ and R₅, which may be the same or different, each represent:

a hydrogen atom,

a halogen atom,

a linear or branched (C₁-C₆)alkyl group,

a linear or branched (C₁-C₆)alkoxy group,

a hydroxy group,

a linear or branched (C₁-C₆)polyhaloalkyl group,

a nitro group,

an amino group optionally substituted by one or two linear or branched (C₁-C₆)alkyl groups,

a group of formula

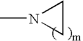

wherein m represents an integer such that 1≦m≦4,

or R₂ with R₃, or R₃ with R₄, or R₄ with R₅, form, together with the carbon atoms carrying them, a 5- to 12-membered, mono- or bi-cyclic, aromatic or non-aromatic group optionally containing 1 or 2 hetero atoms selected from O, S and N, and optionally substituted by one or more identical or different atoms or groups selected from halogen atoms and linear or branched (C₁-C₆)alkyl, hydroxy, linear or branched (C₁-C₆)alkoxy, linear or branched (C₁-C₆)polyhaloalkyl, amino (optionally substituted by one or more linear or branched (C₁-C₆)alkyl groups), nitro, linear or branched (C₁-C₆)acyl and (C₁ -C₂)alkylenedioxy groups,

R₁₀represents a hydrogen atom or a linear or branched (C₁-C₆)alkyl group,

Ar represents an aryl group, heteroaryl group or aryl-(C₁-C₆)alkyl group wherein the alkyl moiety is linear or branched,

to their optical isomers, to addition salts thereof with a pharmaceutically acceptable acid, and to hydrates and solvates thereof.

Among the pharmaceutically acceptable acids there may be mentioned, without implying any limitation, hydrochloric acid, hydrobromic acid, sulphuric acid, phosphoric acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, benzenesulphonic acid, camphoric acid etc.

An aryl group is understood to mean phenyl, biphenylyl, naphthyl or tetrahydronaphthyl, each of those groups being optionally substituted by one or more identical or different atoms or groups selected from halogen atoms and linear or branched (C₁-C₆)alkyl, hydroxy, linear or branched (C₁-C₆)alkoxy, linear or branched (C₁-C₆)polyhaloalkyl, amino (optionally substituted by one or more linear or branched (C₁-C₆)alkyl groups), nitro, linear or branched (C₁-C₆)acyl and (C_{1-C}₂)alkylenedioxy groups.

A heteroaryl group is understood to mean a 5- to 12-membered, mono- or bi-cyclic, aromatic group containing one, two or three hetero atoms selected from oxygen, nitrogen and sulphur, it being understood that the heteroaryl group may be optionally substituted by one or more identical or different atoms or groups selected from halogen atoms and linear or branched (C₁-C₆)alkyl groups, hydroxy groups, linear or branched (C₁-C₆)alkoxy groups, linear or branched (C₁-C₆)polyhaloalkyl groups, and amino groups (optionally substituted by one or more linear or branched (C₁-C₆)alkyl groups). Among the heteroaryl groups, there may be mentioned, without implying any limitation, the groups thienyl, pyridyl, furyl, pyrrolyl, imidazolyl, oxazolyl, isoxazolyl, thiazolyl, isothiazolyl, quinolyl, isoquinolyl and pyrimidinyl.

A nitrogen-containing heterocycle is understood to mean a 5- to 7-membered, monocyclic, saturated group containing one, two or three hetero atoms, one of those hetero atoms being the nitrogen atom and the additional hetero atom(s) optionally present being selected from oxygen, nitrogen and sulphur atoms. Preferred nitrogen-containing heterocycles are the groups pyrrolidinyl, piperidyl, morpholinyl or piperazinyl.

Among the 5- to 12-membered, mono- or bi-cyclic, aromatic or non-aromatic groups optionally containing 1 or 2 hetero atoms selected from O, S and N there may be mentioned, without implying any limitation, the groups phenylene, naphthylene, cyclopentenylene, and the groups of formulae G₁ to G₅:

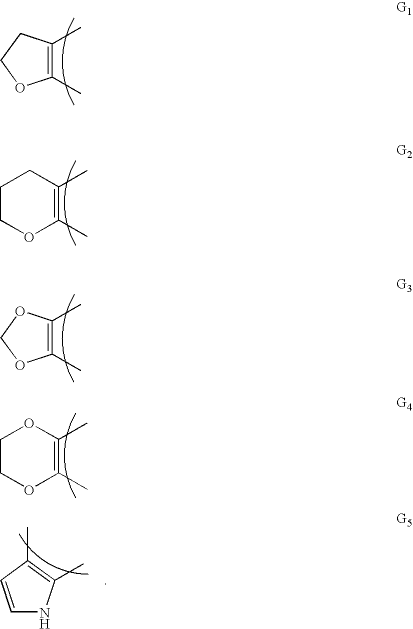

Preferred compounds of formula (I) are those wherein [symbol] represents a double bond.

An advantageous aspect of the invention relates to compounds of formula (I) wherein R₁₀ represents a hydrogen atom.

Another advantageous aspect of the invention relates to compounds of formula (I) wherein R₁₀ represents a linear or branched (C₁-C₆)alkyl group.

Preferred compounds of formula (I) are those wherein

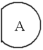

represents a ring system selected from

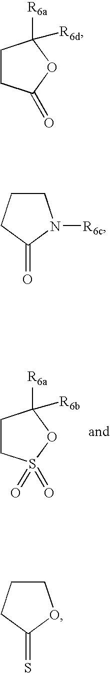

wherein R₆ₐ, R_{6b}, R_{6c} and R_{6d} are as defined for formula (I).

An advantageous aspect of the invention relates to compounds of formula (I) wherein R₂, R₃, R₄ and R₅, which may be the same or different, each represent a hydrogen atom or a linear or branched (C₁-C₆)alkyl group or a linear or branched (C₁-C₆)alkoxy group, or wherein R₂ and R₃, or R₃ and R₄, form, together with the carbon atoms carrying them, a 5- to 12-membered, mono- or bi-cyclic, aromatic or non-aromatic group optionally containing 1 or 2 hetero atoms selected from O, S and N.

Among those compounds, special preference is given to those wherein R₃ and R₄, together with the carbon atoms carrying them, form a group of formula G₃ or G₄ as defined hereinbefore, and those wherein R₂ and R₃, together with the carbon atoms carrying them, form a phenylene group.

An advantageous aspect of the invention relates to compounds of formula (I) wherein Ar represents an aryl group. Among those compounds, special preference is given to those wherein Ar represents an optionally substituted phenyl group.

Another advantageous aspect of the invention relates to compounds of formula (I) wherein Ar represents a heteroaryl group.

Preferred compounds of formula (I) are those wherein R₁ represents a hydrogen atom, a linear or branched (C₁-C₆)alkyl group or an aryl-alkyl group wherein the alkyl moiety is linear or branched (C₁-C₆).

Among the preferred compounds of the invention there may be mentioned more specifically:

6,7-methylenedioxy-9-(3,4,5-trimethoxyphenyl)-2,3,4,9-tetrahydropyrrolo-[3,4-]quinolin-1(1H)-one, its optical isomers, addition salts thereof with a pharmaceutically acceptable acid, and hydrates and solvates thereof;

3,3-dimethyl-6,7-methylenedioxy-9-(3,4,5-trimethoxyphenyl)-4,9-dihydrofuro-[3,4-b]quinolin-1(3H)-one, its optical isomers, addition salts thereof with a pharmaceutically acceptable acid, and hydrates and solvates thereof;

3,3-dimethyl- 1,1-dioxo-6,7-methylenedioxy-9-(3,4,5-trimethoxyphenyl)-4,9-dihydro-3H-[1,2]oxathiolo[4,3-b]quinoline, its optical isomers, addition salts thereof with a pharmaceutically acceptable acid, and hydrates and solvates thereof;

4-benzyl-6,7-methylenedioxy-9-(3,4,5-trimethoxyphenyl)-4,9-dihydrofuro[3,4-b]quinoline-1(3H)-thione, its optical isomers, addition salts thereof with a pharmaceutically acceptable acid, and hydrates and solvates thereof;

6-methoxy-4-methyl-9-(3,4,5-trimethoxyphenyl)-4,9-dihydrofuro[3,4-b]quinoline-1(3H)-thione, its optical isomers, addition salts thereof with a pharmaceutically acceptable acid, and hydrates and solvates thereof;

9-methyl-6,7-methylenedioxy-9-phenyl-4,9-dihydrofuro[3,4-b]quinolin- 1(3H)-one, its optical isomers, addition salts thereof with a pharmaceutically acceptable acid, and hydrates and solvates thereof;

and 9-methyl-6,7-methylenedioxy-9-(3,4,5-trimethoxyphenyl)-4,9-dihydrofuro[3,4-b]quinolin-1(3H)-one, its optical isomers, addition salts thereof with a pharmaceutically acceptable acid, and hydrates and solvates thereof.

The invention relates also to a process for the preparation of compounds of formula I, which process is characterised in that a compound of formula (II):

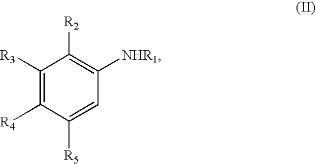

wherein R₁, R₂, R₃, R₄ and R₅ are as defined for formula (I), is reacted with a compound of formula (III):

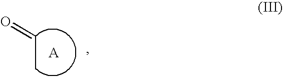

wherein

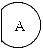

is as defined for formula (I), and with a compound of formula (IV):

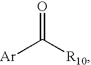

wherein Ar and R₁₀ are as defined for formula (I), to yield the compound of formula (Ia), a particular case of the compounds of formula (I):

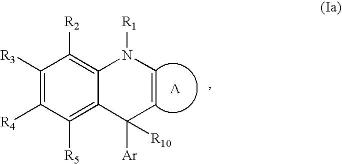

wherein

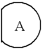

R₁, R₂, R₃, R₄, R₅, R₁₀ and Ar are as defined hereinbefore, which, if desired, is reduced to yield the compound of formula (Ib), a particular case of the compounds of formula (I):

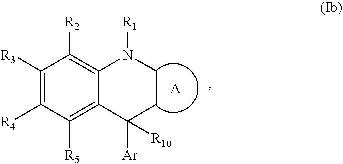

wherein

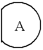

R₁, R₂, R₃, R₄, R₅, R₁₀ and Ar are as defined hereinbefore, the compounds of formulae (Ia) and (Ib) constituting the totality of the compounds of formula (I), which are purified, if necessary, according to a conventional purification technique, are separated, if desired, into their optical isomers according to a conventional separation technique and are converted, if desired, into their addition salts with a pharmaceutically acceptable acid.

The compounds of formula (I) wherein

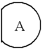

contains a thioxo (═S) group may also be obtained by thionation of the corresponding oxo (═O) group.

Besides the fact that the compounds of the present invention are new, they possess valuable pharmacological properties. They have cytotoxic properties that make them useful in the treatment of cancers.

The invention relates also to pharmaceutical compositions comprising as active ingredient at least one compound of formula (I) together with one or more inert, non-toxic, appropriate excipients. Among the pharmaceutical compositions according to the invention there may be mentioned more especially those that are suitable for oral, parenteral (intravenous, intramuscular or subcutaneous) or nasal administration, tablets or dragees, sublingual tablets, gelatin capsules, lozenges, suppositories, creams, ointments, dermal gels, injectable preparations, drinkable suspensions etc.

The useful dosage can be varied according to the nature and severity of the disorder, the administration route and also the age and weight of the patient and any associated treatments. The dosage varies from 0.5 mg to 2 g per 24 hours in one or more administrations.

The following Examples illustrate the invention but do not limit it in any way.

The starting materials used are known compounds or prepared according to known methods of preparation.

The structures of the compounds described in the Examples have been determined in accordance with the customary spectrometric techniques (infrared, NMR, mass spectrometry).

### EXAMPLE 1

### 2-Methyl-6,7-methylenedioxy-9-(3,4,5-trimethoxyphenyl)-2,3,4,9-tetrahydropyrrolo[3,4-b]quinolin-1(1H)-one

To 10 mmol of 3,4-methylenedioxy-aniline dissolved in ethanol there are added 10 mmol of N-methyl-tetramic acid (the preparation of which is described in Tet. Lett. 1984, 25, 1871) and 10 mmol of 3,4,5-trimethoxybenzaldehyde, and the reaction mixture is then heated at reflux. After cooling, the precipitate obtained is filtered off and then washed and recrystallised to yield the expected product.

### EXAMPLE 2

### 6,7-Methylenedioxy-9-(3,4,5-trimethoxyphenyl)-2,3,4,9-tetrahydropyrrolo[3,4-b]quinolin-1(1H)-one

The expected product is obtained according to the procedure described in Example 1, replacing the N-methyl-tetramic acid by tetramic acid.

Melting point: 244° C.

### EXAMPLE 3

### 3,3-Dimethyl-6,7-methylenedioxy-9-(3,4,5-trimethoxyphenyl)-4,9-dihydrofuro[3,4-]quinolin-1(3H)-one

The expected product is obtained according to the procedure described in Example 1, starting from 3,4-methylenedioxy-aniline, 5,5-dimethyl-tetrahydrofuran-2,4-dione (the preparation of which is described in Chem. Pharm. Bull. 1984, 32, 3724) and 3,4,5-trimethoxybenzaldehyde.

Melting point: >260° C.

### EXAMPLE 4

### 3,3-Dimethyl-1,1-dioxo-6,7-methylenedioxy-9-(3,4,5-trimethoxyphenyl)-4,9-dihydro-3H-[1,2]oxathiolo[4,3-b]quinoline

The expected product is obtained according to the procedure described in Example 1, starting from 3,4-methylenedioxy-aniline, 5,5-dimethyl-2,2-dioxo[1,2]oxathiolan-4-one (the preparation of which is described in Tetrahedron 1997, 17795) and 3,4,5-trimethoxybenzaldehyde.

Melting point: 270-280° C.

### EXAMPLE 5

### 4-Benzyl-6,7-methylenedioxy-9-(3,4,5-trimethoxyphenyl)-4,9-dihydrofuro[3,4-b]quinoline-1(3H)-thione

### Step A: 4-Benzyl-6,7-methylenedioxy-9-(3,4,5-trimethoxyphenyl)-4,9-dihydrofuro[3,4-b]quinolin-1(3H)-one

The expected product is obtained according to the procedure described in Example 1, starting from N-benzyl-3,4-methylenedioxy-aniline, tetronic acid and 3,4,5-trimethoxybenzaldehyde.

Melting point: 236° C.

Elemental microanalysis

| | % C | % H | % N |
|---|---|---|---|
| Calculated: | 68.98 | 5.17 | 2.87 |
| Found: | 68.95 | 5.27 | 2.83 |

### Step B: 4-Benzyl-6,7-methylenedioxy-9-(3,4,5-trimethoxyphenyl)-4,9-dihydrofuro[3,4-b]quinoline-1(3H)-thione

To 10 mmol of the compound obtained in the previous Step, dissolved in a mixture of toluene and dimethyl sulphoxide, there are added 50 mmol of Lawesson's reagent. The reaction mixture is then heated at reflux for one hour and then, after returning to ambient temperature, the solvents are evaporated off and the residue obtained is purified by chromatography over silica (eluant: dichloromethane) to yield the expected product.

Melting point: 129.5° C.

### EXAMPLE 6

### 6,7-Methylenedioxy-9-(3,4,5-trimethoxyphenyl)-4,9-dihydrofuro[3,4-b]quinoline-1(3H)-thione

The expected product is obtained according to the procedure described in Example 5, starting from 3,4-methylenedioxy-aniline, tetronic acid and 3,4,5-trimethoxybenzaldehyde.

### EXAMPLE 7

### 7-(3,4,5-Trimethoxyphenyl)-7,11-dihydrobenzo[h]furo[3,4-b]quinoline-8(10H)-thione

The expected product is obtained according to the procedure described in Example 5, starting from 1-naphthylamine, tetronic acid and 3,4,5-trimethoxybenzaldehyde.

### EXAMPLE 8

### 6-Methoxy-4-methyl-9-(3,4,5-trimethoxyphenyl)-4,9-dihydrofuro[3,4-b]quinoline-1(3H)-thione

### Step A: N-Methyl-3-methoxyaniline

A solution of 3-methoxyaniline (10 mmol) in formic acid (36 ml) is heated at reflux for 1 hour. After removing the excess formic acid, the residue obtained is diluted with water and then extracted with dichloromethane. The combined organic phases are evaporated. The residue obtained is dissolved in ether, and then lithium aluminium hydride (42 mmol) is added at 10° C., in small portions. After stirring for 3 hours at ambient temperature, water and 10 % sodium hydroxide solution are added and the mixture is then filtered over Celite. The filtrate is dried and evaporated and the residue obtained is purified by chromatography over silica, using dichloromethane as eluant, to yield the expected product in the form of an oil.

### Step B: 6-Methoxy-4-methyl-9-(3,4,5-trimethoxyphenyl)-4,9-dihydrofuro[3,4-b]quinoline-1(3H)-thione

The expected product is obtained according to the procedure described in Example 5, replacing the N-benzyl-3,4-methylenedioxyaniline in Step A by the compound obtained in the Step above.

Melting point: 198-200° C.

### EXAMPLE 9

### 9-Methyl-6,7-methylenedioxy-9-phenyl-4,9-dihydrofuro[3,4-b]quinolin-1(3H)-one

100 mmol of acetophenonone are added to 10 mmol of tetronic acid dissolved in trifluoroacetic acid, and the reaction mixture is then heated at reflux for 3 hours. Then, 10 mmol of 3,4-methylenedioxyaniline are added and the solution is then heated at reflux for 2 hours 30 minutes. After evaporating off the solvent under reduced pressure, the residue obtained is purified by chromatography over silica (eluant : dichloromethane/ethyl acetate 90/10) to yield the expected product.

Melting point: >260° C.

Elemental microanalysis:

| | C % | H % | N % |
|---|---|---|---|
| calculated | 71.02 | 4.70 | 4.36 |
| found | 70.93 | 4.84 | 4.20 |

### EXAMPLE 10

### 9-Methyl-6,7-methylenedioxy-9-(3,4,5-trimethoxyphenyl)-4,9-dihydrofuro[3,4-b]quinolin-1(3H)-one

The expected product is obtained according to the procedure described in Example 9, replacing the acetophenone by 3,4,5-trimethoxyacetophenone.

Melting point: >260° C.

Elemental microanalysis:

| | C % | H % | N % |
|---|---|---|---|
| calculated | 64.22 | 5.14 | 3.40 |
| found | 64.15 | 5.33 | 3.34 |

### Pharmacological Study of Compounds of the Invention

### EXAMPLE 11

### In vitro cytotoxicity

Three cell lines were used:

1 murine leukaemia, L1210,

1 human non-small-cell lung carcinoma, A549,

1 human colon carcinoma, HT29.

The cells are cultured in RPMI 1640 complete culture medium containing 10% foetal calf serum, 2 mM glutamine, 50 units/ml of penicillin, 50 μg/ml of streptomycin and 10 mM Hepes, pH=7.4. The cells are distributed on microplates and are exposed to the cytotoxic compounds. The cells are then incubated for 2 days (L1210) or 4 days (A549, HT29). The number of viable cells is then quantified by a colorimetric assay, the Microculture Tetrazolium Assay (Cancer Res. 1987, 47, 939-942).

The results obtained show that the compounds of the invention have in vitro cytotoxicity. By way of example, the compound of Example 5 has an IC₅₀ (concentration of cytotoxic agent which inhibits proliferation of the treated cells by 50%) of 0.19 μM (L1210).

### EXAMPLE 12

### Pharmaceutical Composition

Formula for the preparation of 1000 tablets each
containing 10 mg of active ingredient

| | |
|---|---|
| Compound of Example 5 | 10 g |
| Hydroxypropyl cellulose | 2 g |
| Wheat starch | 10 g |
| Lactose | 100 g |
| Magnesium stearate | 3 g |
| Talc | 3 g |

1. A compound selected from those of formula (I): 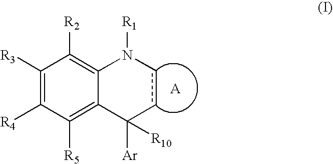
wherein: 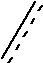
represents a single or double bond, 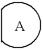
represents a ring system selected from 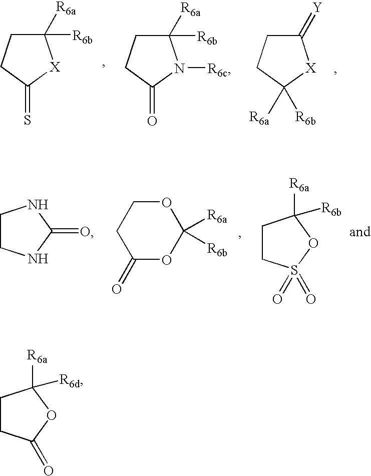
R₆ₐ, R_{6b} and R_{6c}, which may be the same or different, each represent hydrogen or linear or branched (C₁-C₆)alkyl,
R_{6d} represents linear or branched (C₁-C₆)alkyl when R₁₀ represents hydrogen, and a group selected from hydrogen and linear or branched (C₁-C₆)alkyl when R₁₀ represents linear or branched (C₁-C₆)alkyl,
X represents oxygen, sulphur or NR_{6c} wherein R_{6c} represents hydrogen or linear or branched (C₁-C₆)alkyl,
Y represents oxygen or sulphur,
R₁ represents hydrogen or a group selected from
aryl,
heteroaryl,
(C₃-C₈)cycloalkyl,
linear or branched (C₁-C₆)alkyl optionally substituted by aryl, heteroaryl, hydroxy, linear or branched (C₁-C₆)alkoxy, or NR₇ₐR_{7b} wherein R₇ₐ and R_{7b}, which may be the same or different, each represent linear or branched (C₁-C₆)alkyl optionally substituted by hydroxy or amino (itself optionally substituted by one or two linear or branched (C₁-C₆)alkyl), or R₇ₐ and R_{7b}, together with the nitrogen atom carrying them, form a nitrogen-containing heterocycle,
and COR₈ wherein R₈ represents a group selected from:
aryl,
linear or branched (C₁-C₆)alkyl (optionally substituted by NR₇ₐR_{7b} wherein R₇ₐ and R_{7b}, which may be the same or different, each represent linear or branched (C₁-C₆)alkyl optionally substituted by hydroxy or amino (itself optionally substituted by one or two linear or branched (C₁-C₆)alkyl), or R₇ₐ and R_{7b}, together with the nitrogen atom carrying them, form a nitrogen-containing heterocycle),
amino optionally substituted by one or more aryl, heteroaryl, or linear or branched (C₁-C₆)alkyl optionally substituted by NR₇ₐR_{7b} wherein R₇ₐ and R_{7b}, which may be the same or different, each represent linear or branched (C₁-C₆)alkyl optionally substituted by hydroxy or amino (itself optionally substituted by one or two linear or branched (C₁-C₆)alkyl), or R₇ₐ and R_{7b}, together with the nitrogen atom carrying them, form a nitrogen-containing heterocycle,
or OR₉ wherein R₉ represents hydrogen or aryl, or linear or branched (C₁-C₆)alkyl optionally substituted by NR₇ₐR_{7b} wherein R₇ₐ and R_{7b}, which may be the same or different, each represent linear or branched (C₁-C₆)alkyl optionally substituted by hydroxy or amino (itself optionally substituted by one or two linear or branched (C₁-C₆)alkyl), or R₇ₐ and R_{7b}, together with the nitrogen atom carrying them, form a nitrogen-containing heterocycle,
R₂, R₃, R₄ and R₅, which may be the same or different, each represent a group selected from:
hydrogen,
halogen,
linear or branched (C₁-C₆)alkyl,
linear or branched (C₁-C₆)alkoxy,
hydroxy,
linear or branched (C₁-C₆)polyhaloalkyl,
nitro,
amino optionally substituted by one or two linear or branched (C₁-C₆)alkyl, 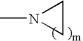
wherein m is 1 to 4 inclusive, or R₂ with R₃, or R₃ with R₄, or R₄ with R₅, form, together with the carbon atoms carrying them, a 5- to 12-membered, mono- or bi-cyclic, aromatic or non-aromatic group optionally containing 1 or 2 hetero atoms selected from O, S and N, and optionally substituted by one or more identical or different atoms or groups selected from halogen, linear or branched (C₁-C₆)alkyl, hydroxy, linear or branched (C₁-C₆)alkyl, linear or branched (C₁-C₆)polyhaloalkyl, amino (optionally substituted by one or more linear or branched (C₁-C₆)alkyl), nitro, linear or branched (C₁-C₆)acyl and (C₁-C₂)alkylenedioxy,
R₁₀ represents hydrogen or linear or branched (C₁-C₆)alkyl,
Ar represents aryl, heteroaryl or aryl-(C₁-C₆)alkyl wherein the alkyl moiety is linear or branched,
its optical isomers, addition salts thereof with a pharmaceutically acceptable acid, and hydrates and solvates thereof,
aryl means phenyl, biphenylyl, naphthyl or tetrahydronaphthyl, each of those groups being optionally substituted by one or more identical or different atoms or groups selected from halogen, linear or branched (C₁-C₆)alkyl, hydroxy, linear or branched (C₁-C₆)alkoxy, linear or branched (C₁-C₆)polyhaloalkyl, amino (optionally substituted by one or more linear or branched (C₁-C₆)alkyl), nitro, linear or branched (C₁-C₆)acyl and (C₁-C₂)alkylenedioxy,
heteroaryl means a 5- to 12-membered, mono- or bi-cyclic aromatic group containing one, two or three hetero atoms selected from oxygen, nitrogen and sulphur, it being understood that the heteroaryl group may be optionally substituted by one or more identical or different atoms or groups selected from halogen, linear or branched (C₁-C₆)alkyl, hydroxy, linear or branched (C₁-C₆)alkoxy, linear or branched (C₁-C₆)polyhaloalkyl, and amino (optionally substituted by one or more linear or branched (C₁-C₆)alkyl),
and a nitrogen-containing heterocycle means a 5- to 7-membered, monocyclic, saturated group containing one, two or three hetero atoms, one of those hetero atoms being nitrogen and the additional hetero atom(s) optionally present being selected from oxygen, nitrogen and sulphur.

2. A compound of claim 1, wherein 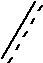
Represents a double bond.

3. A compound of claim 1, wherein R₁₀ represents hydrogen.

4. A compound of claim 1, wherein R₁₀ represents linear or branched (C₁-C₆)alkyl.

5. A compound of claim 1, wherein 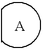
represents a ring system selected form 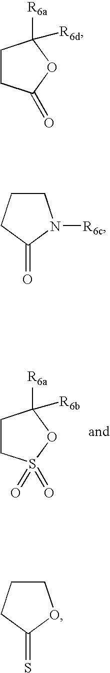
wherein R₆ₐ, R_{6b}, R_{6c} and R_{6d} are as defined in claim 1

6. A compound of claim 1, wherein R₂ and R₃, or R₃ and R₄, together with the carbon atoms carrying them, form a 5- to 12-membered, mono- or bi-cyclic, aromatic or non-aromatic group optionally containing 1 or 2 hetero atoms selected from O, S and N.

7. A compound of claim 6, wherein R₃ and R₄,together with the carbon atoms carrying them, form a group of formula G₃ or G_{4:}

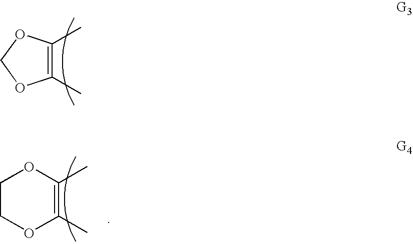

8. A compound of claim 6, wherein R₂ and R₃, together with the carbon atoms carrying them, form a phenylene group.

9. A compound of claim 1, wherein R₂, R₃, R₄ and R₅, which may be the same or different, each represent hydrogen, linear or branched (C₁-C₆)alkyl or linear or branched (C₁-C₆)alkoxy.

10. A compound of claim 1, wherein Ar represents aryl.

11. A compound of claim 10, wherein Ar represents optionally substituted phenyl.

12. A compound of claim 1, wherein Ar represents heteroaryl.

13. A compound of claim 1, wherein R₁ represents hydrogen, linear or branched (C₁-C₆)alkyl or aryl-alkyl wherein the alkyl moiety is linear or branched (C₁-C₆).

14. A compound of claim 1, which is selected from
6,7-methylenedioxy-9-(3,4,5-trimethoxyphenyl)-2,3,4,9-tetrahydropyrrolo[3,4-b]quinolin-1(1H)-one,
3,3-dimethyl-6,7-methylenedioxy-9-(3,4,5-trimethoxyphenyl)-4,9-dihydrofuro[3,4-b]quinolin- 1 (3H)-one
3,3-dimethyl-1,1-dioxo-6,7-methylenedioxy-9-(3,4,5-trimethoxy-phenyl)-4,9-dihydro-3H-[1,2]oxathiolo[4,3-b]quinoline,
4-benzyl-6,7-methylenedioxy-9-(3,4,5-trimethoxyphenyl)-4,9-dihydrofuro[3,4-b]quinoline-1(3H)-thione,
6-methoxy-4-methyl-9-(3,4,5-trimethoxyphenyl)-4,9-dihydrofuro[3,4-b]quinoline-1(3H)-thione
9-methyl-6,7-methylenedioxy-9-phenyl-4,9-dihydrofuro[3,4-b]quinolin- 1(3H)-one, 9-methyl-6,7-methylenedioxy-9-(3,4,5-trimethoxyphenyl)-4,9-dihydrofuro[3,4-b]quinolin- 1(3H)-one
its optical isomers, addition salts thereof with a pharmaceutically acceptable acid, and hydrates and solvates thereof.

15. A pharmaceutical composition comprising as active principle an effective amount of a compound of claim 1, together with one or more pharmaceutically acceptable excipients or vehicles.

16. A method for treating a living animal body afflicted with cancer, comprising the step of administering to the living animal body an amount of a compound of claim 1 which is effective for alleviation of the condition.
